(19) United States
(12) Patent Application Publication
(10) Pub. No.: US 2025/0032305 A1
(43) Pub. Date: Jan. 30, 2025

(54) Adult Male Disposable Incontinence System

(71) Applicant: Robert Brandeis Bennett, Apollo Beach, FL (US)

(72) Inventor: Robert Brandeis Bennett, Apollo Beach, FL (US)

(21) Appl. No.: 18/430,646

(22) Filed: Feb. 2, 2024

Related U.S. Application Data
(60) Provisional application No. 63/515,132, filed on Jul. 24, 2023.

(51) Int. Cl. A61F 5/453; A61F 5/44; A61F 13/15; A61F 13/471; A61F 13/53

(52) U.S. Cl. CPC A61F 5/453; A61F 5/4401; A61F 5/4408; A61F 13/471; A61F 2013/15146; A61F 2013/530007; A61F 2013/530489

(57) ABSTRACT

Disclosed embodiments relate to a collection device for bodily fluids comprising a tubular material with a first end and second end. A portion of the first end is configured to be secured to a user and the tubular material may be folded over itself to form a first sheath. In some applications, the fluid collection devices described may be secured to a user using stretch tape and a hook and loop connector.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants make no claim to any trademarks referenced.

### BACKGROUND OF THE INVENTION

### Field of the Invention

The invention relates to a urinary incontinence device. Urinary incontinence is defined as the loss of bladder control permitting urine to inadvertently leak from the bladder. It has been estimated that up to 35% of noninstitutionalized older adults are incontinent and greater than 50% of nursing home occupants are incontinent with regards to urine discharges. Incontinence is a problem that is also associated with prostate surgery and treatment for prostate cancer.

Urinary incontinence is a costly medical problem both in health care costs and poor patient outcomes. Annually, millions of health dollars are spent to diagnose the causes of incontinence and to provide medical treatment either with medications, surgery and/or various known devices.

### DESCRIPTION OF RELATED ART

Currently the state of the art includes various underwear and condoms that attempt to capture the urine being discharged.

The current status of devices such as disposable underwear is that they only partially absorb the urine, and they are commonly used to maintain some semblance of hygiene. In spite of the use of disposable underwear, clothes and bed linens require more frequent laundering as they are frequently soaked through wetting. Due to the incontinence, the patient's skin is frequently wet, in an acid environment, resulting in skin breakdown and its complications in bedridden and institutionalized individuals.

Frequently, due to caregiver frustration with the effects of incontinence and the lack of a reliable urine external collection system, indwelling tubes are placed into the bladder (i.e., a foley catheter). These indwelling bladder catheters inevitably become colonized with bacteria causing frequent infections that are often life threatening in elderly or disabled patients.

Disposable diapers and briefs/underwear are the most common solution to urinary incontinence in the general population as well as hospitalized and institutionalized patients.

External male urine collection systems are infrequently used because the currently available systems are prone to detachment and wetting. These devices are generally ineffective and uncomfortable for the wearer. Currently the use of external male urine collection systems are not sufficiently dependable to allow its use for multiple hours without falling off or becoming increasingly uncomfortable for the wearer.

The most common external collection device used for hospitalized patients is the external condom catheter, comprising a tube connected to an end of a condom collection unit that is attached to the penis with an adhesive or pressure. The collection unit rarely functions properly because the penis constantly changes size during the day and the condom collection assembly falls off, wasting caregiver time and institutional monies. A dry genital area cannot be maintained when the condom is displaced from its initial position and therefore leaks urine from the collection unit. Another approach maintains the condom collection unit in place with an elastic belt that fits around the patient's waist and/or legs. Force exerted by the elastic retains the collection unit in place.

Another urine collection approach comprises a custom brief further comprising a condom-like catheter disposed within a pocket formed in the brief. The urine exits the condom catheter into a collection bag. This unit is expensive and, like the other condom-type units described above, fails when the penis contracts.

Due to the various described disadvantages for the catheter-type urine collection systems, a disposable brief or underwear made from a non-woven material is therefore the most common approach for managing urinary incontinence, as well as fecal incontinence. However, in spite of its high absorbency properties the diaper or brief eventually becomes wet. Prolonged exposure to wet underwear can cause skin breakdown and multiple other ensuing complications. There is therefore a need for a comfortable, reliable, disposable external male urine collection system.

### SUMMARY OF THE INVENTION

In a first implementation of the invention a male urine collection device for attachment to a penis is disclosed. The instant invention comprises of a tubular device similar to a sock or condom that fits over the male penis and is secured to the penis with a fastener such as adhesive tape, stretch tape, hook, and loop fastener such as Velcro®. The reason that the instant invention stays attached to the penis is because the end that attaches to the penis is small enough so it cannot slip off the penis when securely fastened. The collection device can also comprise of a penis attachment device disposed in the opening for receiving and restraining a penis; a urine collection assembly wherein the penis extends into the urine collection assembly from the attachment device and wherein the urine assembly collection device receives urine released from the penis. The urine collection assembly is comprised of liquid absorbing materials that surround the penis so that any discharge can be quickly absorbed by the liquid absorbing materials. The urine collection assembly also has a pleated side mechanism that allows the urine to travel up the side of the urine collection device so that the absorbent material contained in the urine collection assembly is uniformly wetted as urine enters the urine collection assembly.

### BRIEF DESCRIPTION OF DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1, shows a perspective view of a first embodiment of the instant invention.

FIG. 2 is a plan view of the instant invention shown in FIG. 1 showing an incontinence device shaped in a tabular fashion with a closed distal end and an open proximal end.

FIG. 3 is an end view of the instant invention shown in FIG. 1 showing the enclosed end of the incontinence device.

FIG. 4 is an end view of the instant invention shown in FIG. 1 showing the open end of the incontinence device for receiving the penis.

FIG. 5 is a view of an embodiment of the urine collection pre-assembly.

FIG. 6 shows the absorbent filler placed in the nonabsorbent retainer to form the absorbent core.

FIG. 7 shows a view of the nonabsorbent retainer folded over to encase the absorbent filler.

FIG. 8 shows a view of the urine collection pre-assembly and outer sheath.

FIG. 9 shows a second embodiment of the instant invention showing an alternative design that is configured to hold a large amount of urine and be less intrusive for the patient.

FIG. 10 shows the absorbent filler of the alternative design of FIG. 9.

FIG. 11 shows a plan view of the cladding layer of the alternative design of FIG. 9.

FIG. 12 shows an alternative cladding layer that is formed from the alternative outer layer of alternative cladding section and alternative stretch section.

FIG. 13 shows the alternative cladding layer and the absorbent core.

FIG. 14 shows the alternative incontinence device folded over and seams sealed.

FIG. 15 shows a view of section A-A in FIG. 12.

FIG. 16 shows a line drawing perspective view of an alternative incontinence device.

FIG. 17 shows a line drawing plan view of the alternative incontinence device shown in FIG. 16.

FIG. 18 shows a line drawing side view of the alternative incontinence device shown in FIG. 16.

FIG. 19 shows a line drawing end view from open proximal end of the alternative incontinence device shown in FIG. 16.

FIG. 20 shows a rendering of a perspective view of the incontinence device of the alternative incontinence device shown in FIG. 16

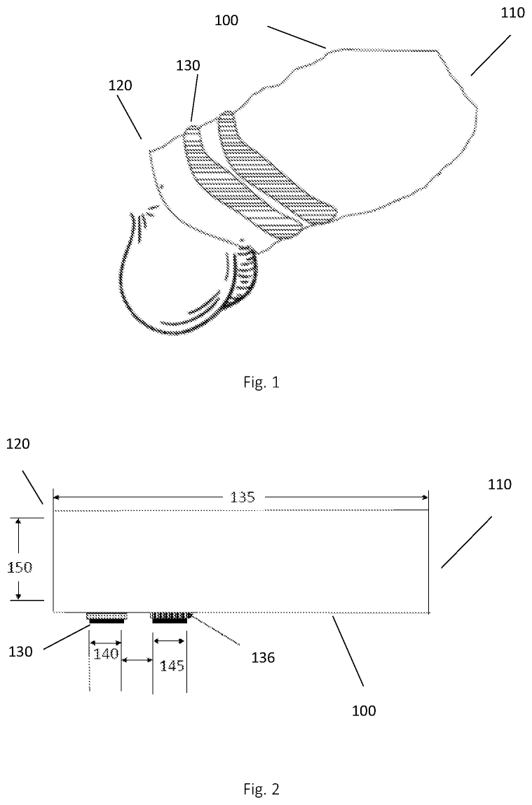

With respect to the FIG. 1-20, corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. Therefore, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps, or acts are in some way inherently mutually exclusive.

As this invention is susceptible to embodiments of many different forms, it is intended that the present disclosure be considered as an example of the principles of the invention and not intended to limit the invention to the specific embodiments shown and described.

The terms penis as used in this specification is a part of the human anatomy used to discharge urine.

The term pre-assembly as used in the specification is meant to mean a part of the production process such as an initial assembly of certain component groups that forms part of an assembled final device.

The instant invention is a liquid collection device and more specifically a urine collection device that is attached to a male penis. The instant invention comprises a sock or condom that fits over the male penis and is secured to the penis with a fastener such as adhesive tape, stretch tape, hook, and loop fastener such as Velcro® or any other suitable attachment method. The collection device can also comprise of a penis attachment device disposed at the opening of the collection device for receiving and restraining a penis; a urine collection assembly wherein the penis extends into the urine assembly collection assembly from the attachment device and wherein the urine assembly collection device receives urine released from the penis. The urine collection assembly is comprised of liquid absorbing materials that surround the penis so that any discharge can be quickly absorbed by the liquid absorbing materials.

An alternative method of securing the sock or condom includes having at least one strap connected to the sock or condom and one strap could be placed around the waist, one strap placed around the right thigh, and one strap placed around the left thigh to provide three points of attachment to hold the sock or condom to the user's body. The straps can also be limited to only one or more straps if needed for comfort of the user. The three-strap system provides an attachment means that allows the sock or condom to be attached to the penis securely because the penis in most cases is below the waist and above the crotch of the thighs.

The sock or condom comprises a pre-assembly formed by placing an absorbent material in a nonabsorbent retainer formed in the shape of a tube. The nonabsorbent retainer can be formed from a material that can transmit the urine or fluid and provide a soft compliant support to hold the absorbent material and not cause chaffing of the penis. Typical material for use as the nonabsorbent retainer include but are not limited to polyester or polypropylene non-woven fabric materials.

This first pre-assembly is then attached to a cladding layer formed from non-porous material such as polypropylene or polyester stretch fabric, polypropylene or polyester fabric or other non-porous material that does not transmit liquids and provides a barrier and holding structure for the instant invention to contain the absorbent material and any urine discharged from a penis.

The absorbent material, also described as the absorbent core, is the most important structural component in the instant invention. It is the main absorption layer. A key design factor is that it is required to absorb urine quickly and spread urine to the whole absorption core by the use of diversion, channeling, and core absorption. The absorbent core can incorporate into the design an absorbent resin (SAP). An SAP is designed to quickly absorb and lock urine or other liquids. The SAP makes the absorption of liquid a non-reversable osmosis process.

The absorbent core can also contain absorbent villous pulp that is used to absorb the liquid or urine discharged from the penis.

In another embodiment the absorbent core can also contain absorbent villous pulp or fluff pulp with superabsorbent polymer (SAP) (also called slush powder). SAPs are a water-absorbing hydrophilic homopolymer or copolymer that can absorb and retain substantial amounts of a liquid relative to its own mass. SAP or Water-absorbing polymers, which are classified as hydrogels when mixed, absorb aqueous solutions through hydrogen bonding with water molecules.

There are three main classes of SAPs, and the instant invention can utilize any suitable combination with the fluff pulp or absorbent layer without the fluff pulp. The three main classes of SAPs are:
1. Cross-linked polyacrylates and polyacrylamides.
2. Cellulose- or starch-acrylonitrile graft copolymers.
3. Cross-linked maleic anhydride copolymers.

An alternative absorbent core that does not have fluff pulp can also be used to form the absorbent core. The structure of this alternative absorbent core combines dust-free paper with porous fluffy nonwovens and SAP. The result is that the absorbent core contains a large amount of SAP, and the absorbent volume of the instant invention becomes larger.

The instant invention absorbent core in a first embodiment can be made with a villous pulp (fluff pulp) and other materials. In one embodiment the absorbent core of the instant invention is mainly composed of nonabsorbent retainer, SAP, and villous pulp (fluff pulp). The SAP and villous pulp (fluff pulp) are mixed in a certain proportion to form SAP/villous pulp (fluff pulp) absorbent filler. The absorbent filler is then placed in the nonabsorbent retainer to form the absorbent core. The absorbent core is attached to the cladding layer to form the fabric assembly of the instant invention.

The nonabsorbent retainer can be made of a tissue paper or non-woven materials or multi-layer composite materials. Typical materials for use as the nonabsorbent retainer include but are not limited to pulp-based tissue, rayon, polyurethane, polypropylene, Air-laid paper, polyethylene terephthalate, fiberglass and polyethylene based woven and nonwoven fabrics. The nonabsorbent retainer usually has the function of transmissibility of the urine and ventilation, while the cladding layer performs the function of anti-seepage and liquid barrier. The nonabsorbent retainer must also provide a smooth and non-chafing surface to protect the skin of the penis.

The instant invention absorbent core in a second embodiment can be made with a composite of SAP and porous fluffy non-woven material. The SAP and porous fluffy non-woven material forms a compact that is sandwiched between a nonabsorbent retainer and the cladding layer to form the fabric assembly of the instant invention.

As noted, wherever bonding or an attachment process is needed for attaching any of the parts of the instant invention to another part of the instant invention the typical process used can include but are not limited to:
a. adhesive;
b. heat sealer;
c. calendaring through heated rollers;
d. ultrasonic pattern bonding;
e. ultrasonic sewing;
f. needle punching/needle felting;
g. chemical bonding; and
h. melt-blown bonding.

These bonding processes can be used for sealing and not limited to the pleats of the instant invention, attaching the at least one retaining device to the tubular body, attaching the absorbent core, attaching the nonabsorbent retainer to the cladding layer or attaching the absorbent core to the tubular body.

The instant invention is configured such that the opening that has the connecting device such as a hook tab works best if the collar is constructed from a waterproof stretch material without fluff or SAP. It can optimally have a collar made of stretch material that is elasticized with the non-stretch latching device such as a hook tab attached so that the collar/tab stretches, wraps, and holds the instant invention to the penis to make it secure and prevent it from being dislodged during use. Ideally one collar/tab can suffice to hold the instant invention to the penis, but the instant invention can also be configured to have more than one tab and latching device. The instant invention can also comprise of different cross sections and one preferred embodiment has a penis attachment section that is reduce in size so that it fits tightly above the head of the penis. When a user puts on the instant invention the user stretches the collar/tab to fasten the instant invention to the penis and make it tight enough to secure it with the latching device connector such as the hook tab.

The instant invention creates a container for the fluff and SAP that forms an absorbent and waterproof holder or vessel below the head of the penis and a shaft interface with at least one stretch collar/tab with hook tab to attach the shaft interface securely above the head of the penis to secure the instant invention to the penis. The connecting device, connector or hook tab can be fashioned from an alternative fastening system similar to the stretch tape or other suitable connecting and sealing mechanisms. The instant invention envisions a number of different connection methods such that the user can be provided with an alternative fastening system if desired to accommodate different tactile limitations and make it easier to install or remove. These include but are not limited to clips, tabs, belts, or elastic bands.

An alternate design can have a sealing ring that acts as an O-ring to secure the instant invention to the penis and seal it to the penis shaft.

The instant invention can be manufactured in assorted sizes to accommodate a range of urine collection. The instant invention can be constructed to absorb urine from minor leakage events to overnight leakage events. The attachment mechanism would remain the same and the primary difference is the size of the fluff filled receptacle would be different depending on the application.

Currently, the products available to accommodate minor or major leakage events are pads of varying sizes and shapes.
a. The advantage of the instant invention is the ability to have differentiating designs that covers the various urine leakage problems encountered by the patient population. The design of the instant invention provides a self-contained and absorbent device. The instant invention design helps prevent accidents or leakage from occurring during sleep or when engaged with daytime activities. The instant invention eliminates the minor leakage problems incurred when using pads that are primarily used by patients during the day. Leakage when using pads can cause embarrassment and odor. This problem is eliminated by using the instant invention. Pads are not self-contained and therefore disposal is not an odor and moisture free event such as with the instant invention.

The instant invention remains in position, during sleep, during moderate and even strenuous and extreme physical activity due to the narrow neck that attaches proximal to the end of the penis. This provides a secure and leak-proof attachment. The part of the instant invention distal from the attachment to the penis is designed to hold urine to accommodate a variety of activities including and not limited to sleeping, moderate exercise and daily activity such as walking, sitting and various daily activities such as work or school.

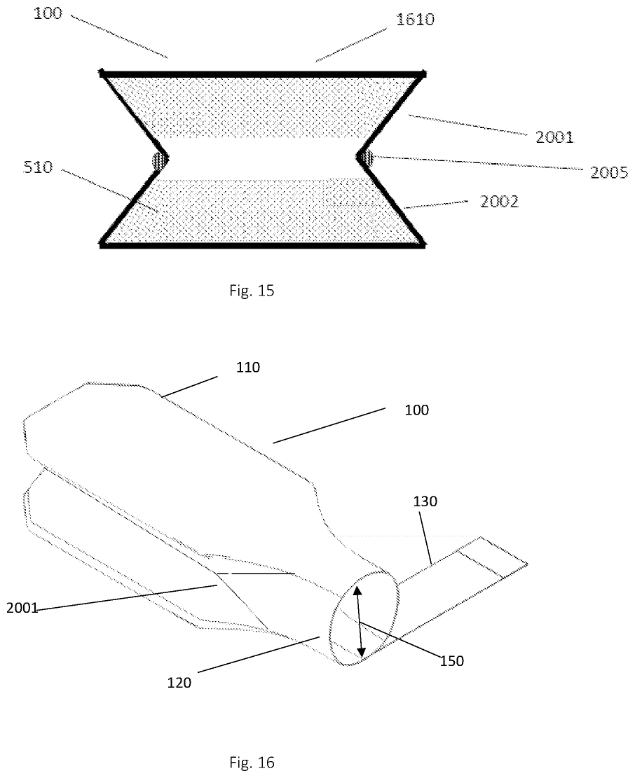

In an alternative design the instant invention is formed from a folded assembly of outer alternative cladding layer that forms the alternative outer layer of alternative cladding section and alternative stretch section. The alternative cladding layer encases an absorbent core. The alternative cladding layer has an alternative transition that is designed to form a suitable connection to alternative stretch section. The alternative cladding section transitions with an alternative length that can range from 0.1-5 inches. The alternative stretch section can have an alternative stretch section length and alternative stretch section width that is suitable for the connection for the two pieces. The alternative cladding layer has an alternative cladding layer length that can range from 1-15 inches and alternative stretch section width can range from 0.25-12 inches. The alternative cladding layer forms a pleat shown in FIG. 15 that allows the alternative outer layer of cladding section to open and close. This opening and closing action forms a channel that allows urine or discharge from the penis to travel up the alternative cladding layer and spread to the absorbent core so that the absorbent material contained in the urine collection assembly is uniformly wetted as urine enters the urine collection assembly. Alternatively, a channel could be formed from a tube inserted in the sides of the instant invention that could transfer urine or other liquid up the sides of the instant invention. The tube would have breaches in the tube to allow the urine to distribute up the tube and fill the absorbent material adjacent to the tube and breaches in the tube for feeding urine to distinct levels. Once the local absorbent material adjacent to the tube was full then filling the next level of absorbent material adjacent to the tube would be accomplished by urine moving up the tube to the next level.

The pleat or channel allows the urine or discharge from the penis to fill the absorbent core evenly and mitigates the problem of urine or discharge from the penis leaking from the open proximal end. The pleat in the final assembly is formed by folding the alternative outer layer of alternative cladding section and alternative stretch section that encases the absorbent core and sealing the free edges that are the half pleat thereby forming a full pleat. The half pleat has a width and edge. The half pleat forms one half of the full pleat as shown in FIG. 15.

The incontinence device of the instant invention with the pleat formed by cladding layer and the sealing of the edge of the half pleat along the edges is critical to the design due to the convenient formation of the channel. The pleat is sealed by using a suitable adhesive. Once sealed the cladding layer encloses the absorbent core and forms a watertight seal with a channel running the entire length of the pleat.

The instant invention can further be described as an incontinence device for males, comprising:
a. a tubular body having a closed distal end and an open proximal end for receiving a penis;
b. at least one retaining device for securing the tubular body to the penis;
c. an absorbent core internal to the tubular body for absorbing urine.
d. said tubular body having a non-porous outer surface that forms at least one channel on the side of said incontinence device; and
e. said at least one channel is formed by sealing two edges of said non-porous outer surface such that said non-porous outer surface forms a pleat and said pleat forms a channel.

The incontinence device of instant invention, wherein the at least one retaining device comprises a fastener selected from the group consisting of adhesive tape, stretch tape, hook, and loop fastener.

The incontinence device of instant invention, wherein the absorbent core comprises a nonabsorbent retainer and an absorbent filler.

The incontinence device of instant invention, wherein the absorbent filler comprises a mixture of superabsorbent polymer (SAP) and villous pulp.

The incontinence device of instant invention, wherein the tubular body is formed from a cladding layer comprising a non-porous material.

The incontinence device of instant invention, further comprising a plurality of retaining devices for securing the tubular body to the penis.

The incontinence device of instant invention, wherein the tubular body is configured to hold a volume of urine ranging from minor leakage events to overnight leakage events.

The incontinence device of instant invention, wherein the tubular body is configured to remain in position during sleep and during moderate to strenuous physical activity.

The incontinence device of instant invention, wherein the tubular body is configured to prevent leakage of urine during use.

The incontinence device of instant invention, wherein the tubular body is configured to be disposable after use.

The incontinence device of instant invention, wherein the tubular body is configured for multiple uses with a washable or replaceable absorbent core.

The instant invention can further be described as an incontinence device for males, comprising:
a. a tubular body having a closed distal end and an open proximal end for receiving a penis;
b. at least one retaining device for securing the tubular body to the penis;
c. an absorbent core internal to the tubular body for absorbing urine.
d. said tubular body having a non-porous outer surface that forms at least one channel on the side of said incontinence device; and
e. said at least one channel is formed by sealing two edges of said non-porous outer surface that forms at least one channel on the side of said incontinence device; and
f. the tubular body is configured to be disposable after use.

The incontinence device for males of instant invention wherein the tubular body is selected from the group consisting of latex, silicone, and a flexible plastic material.

The incontinence device of instant invention, wherein the at least one retaining device comprises a fastener selected from the group consisting of adhesive tape, stretch tape, hook, loop fastener, straps, belts, buckle, a snap and magnetic closure device.

The incontinence device of instant invention, wherein the absorbent core comprises a nonabsorbent retainer and an absorbent filler and the absorbent filler is selected from the group comprising of cotton, cellulose, and a superabsorbent polymer.

The incontinence device of instant invention, wherein the absorbent filler comprises a mixture of superabsorbent polymer (SAP) and villous pulp, rayon, and bamboo fiber.

The incontinence device of instant invention, wherein the tubular body is formed from a cladding layer comprising a non-porous material and the cladding layer is made from a breathable material to promote comfort and reduce skin irritation.

The incontinence device of instant invention, further comprising a plurality of retaining devices for securing the tubular body to the penis and the retaining devices are adjustable to accommodate different penis sizes and shapes.

The incontinence device of instant invention, wherein the tubular body is configured to hold a volume of urine ranging from minor leakage events to overnight leakage events and the tubular body may include a graduated scale to indicate the volume of urine collected.

The incontinence device of instant invention, wherein the tubular body is configured to remain in position during sleep and during moderate to strenuous physical activity.

Referring now to the drawings FIG. 1-20, and more particularly to FIG. 1, there is shown a perspective view of the instant invention. The incontinence device 100 is shaped in a tabular fashion with a closed distal end 110 and an open proximal end 120. The internal diameter 150 shown in FIG. 4 depends on the size of the penis to be used with the instant invention. The incontinence device 100 has at least one retaining device 130.

The incontinence device 100 is shaped in a tabular fashion and it comprises of a long tubular body, a closed distal end 110, an open proximal end 120 for receiving a penis, at least one retaining device 130 and an absorbent core internal to a nonabsorbent retainer.

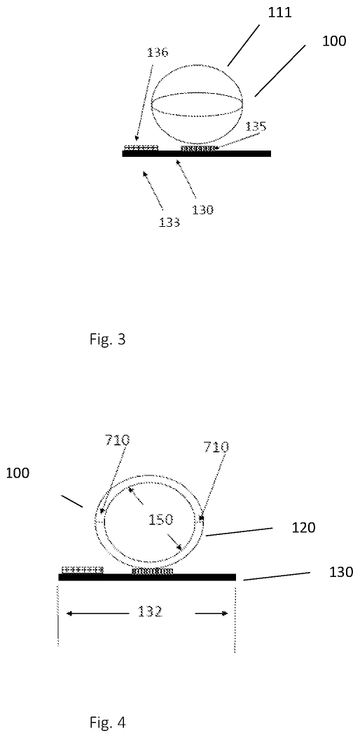

FIG. 2 is a plan view of the instant invention showing incontinence device 100 shaped in a tabular fashion with a closed distal end 110 and an open proximal end 120 and at least one retaining device 130. The length 135 is typically at least 2 inches and as long as 9 inches. However, the length is selected depending on the size of the penis to be used with the instant invention. The internal diameter 150 shown in FIG. 4 of the incontinence device 100 can be from 0.5 inches to 4 inches in diameter depending on the size of the penis to be used with the instant invention. The radius 111 shown in FIG. 3 of the closed end 110 depends on the internal diameter and the thickness of the shell of the incontinence device 100 and can be from 0.2 5 inches to 2 inches in diameter depending on the size of the penis to be used with the instant invention.

The width of the retaining device 130 shown as width 1 element 140 and width 2 element 145 can be as thin as 0.25 inches and as wide as 1 inch and the width and length depend on the size of the penis to be used with the instant invention. The retaining devices 130 are attached to the incontinence device 100 using attachment method 136 that can be selected from either heat activated adhesive, adhesive, or stitching either thread based or ultrasonic bonding.

FIG. 3 is an end view of the instant invention showing the enclosed end 110 of the incontinence device 100 having radius 111.

FIG. 4 is an end view of the instant invention showing the open end 120 of the incontinence device 100 for receiving the penis. The incontinence device 100 can have 1 or more seams 710 depending on the design of the incontinence device 100. The retaining devices length 132 of retaining devices 130 can be from 2 inches to 8 inches in length and the length depends on the size of the penis to be used with the instant invention. The retaining devices 130 are secured to a user using stretch tape, hook and loop connectors or a combination of stretch tape 133 and hook and loop connectors 136 shown in FIG. 3. If the hook and loop connectors are used, they are positioned on the retaining devices 130 and preferably on at least one end of the retaining device 130.

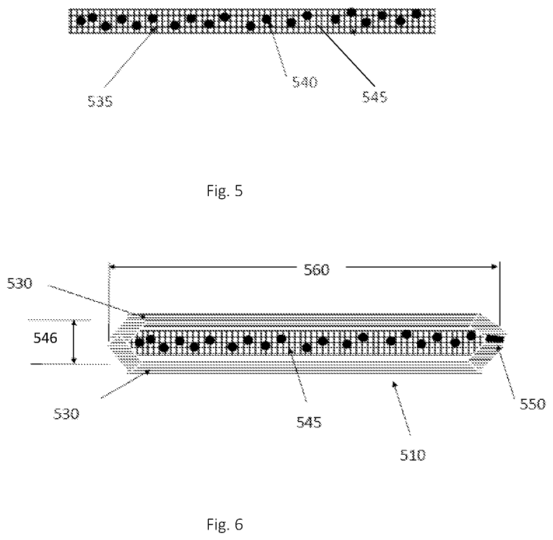

FIG. 5 is a view of a first embodiment of the urine collection pre-assembly. The absorbent core 510 shown in FIG. 8 of the first embodiment is made with a villous pulp (fluff pulp) 535. The absorbent core 510 is mainly composed of nonabsorbent retainer 530 shown in FIG. 6, SAP 540, and villous pulp (fluff pulp) 535. The SAP 540 and villous pulp (fluff pulp) 530 are mixed in a certain proportion to form SAP/villous pulp (fluff pulp) absorbent filler 545.

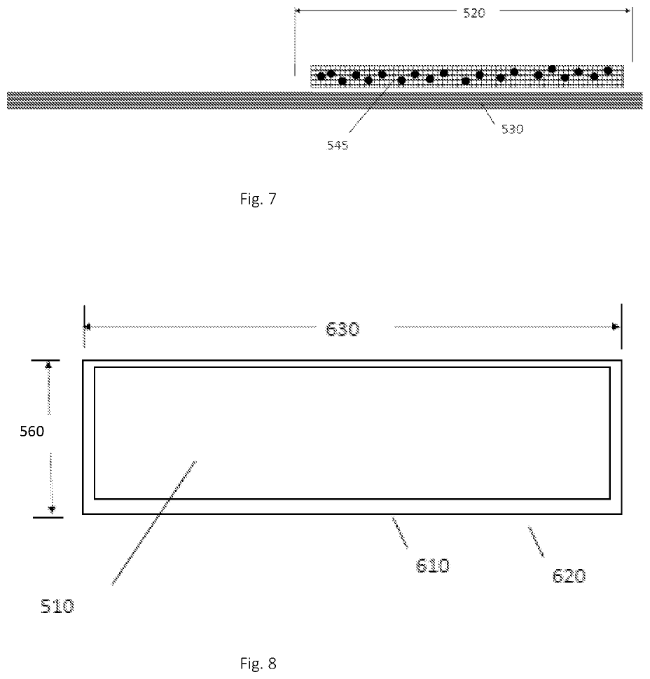

FIG. 6 shows the absorbent filler 545 is then placed in the nonabsorbent retainer 530 to form the absorbent core 510 shown in FIG. 7. The nonabsorbent retainer 530 has a nonabsorbent retainer length of 520 shown in FIG. 7 that depends on the final size of the absorbent core 510 desired. The thickness of the absorbent filler 545 is 546 and depends on the amount of absorbent material needed for the absorbent core 510.

As shown in FIG. 7 the nonabsorbent retainer 530 is folded over to encase the absorbent filler 545 and the nonabsorbent retainer seam 550 shown in FIG. 6 is either bonded, sewn, welded, or made with an adhesive. Nonabsorbent retainer width 560 shown in FIG. 8 depends on the size of the final assembly desired. The nonabsorbent retainer width 520 for a single size incontinence device 100 shown in FIG. 1 can be from 2 inches to 10 inches depending on the final configuration and the size of the penis to be used with the instant invention.

FIG. 8 is a view of the urine collection pre-assembly and outer sheath. The absorbent core 510 is attached to the cladding layer 610 to form the fabric assembly 620 of the instant invention having nonabsorbent retainer width 561. The urine collection pre-assembly length 630 can be from 3 inches to 14 inches depending on the final configuration and the size of the penis to be used with the instant invention. The nonabsorbent retainer width 561 can be from 1 inch to 8 inches depending on the final configuration and the size of the penis to be used with the instant invention.

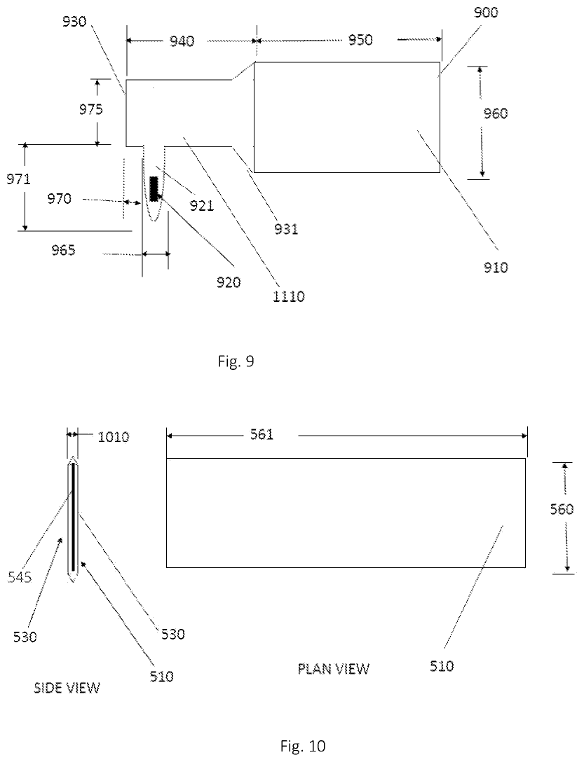

FIG. 9 shows an alternative design that is configured to hold a large amount of urine and be less intrusive for the patient. The assembled device 900 is comprised of cladding section 910 and stretch section 1110 that is formed to create a tube 930 designed to fit over a penis. Cladding section 910 is formed from a non-absorbent material and contains an absorbent filler to collect and hold urine. The cladding section 910 is attached to the stretch section 1110 that is configured to attach to the end of a penis. The stretch section 1110 is designed so as it seals the stretch section 1110 to the penis to prevent fluid escaping from the assembled device 910. Stretch section 1110 has a stretch section width of 975 that can range from 0.25-12 inches and has a stretch section length 940 that can range from 0.25-9 inches.

An assembled device tab 921 is used to seal the assembled device 900 to the penis (not shown) using the hook connector 920 that is placed so as to hold the assembled device 900 to the penis. The instant invention envisions at least one assembled device tab 921 so the embodiment can have more than one assembled device tab 921. The assembled device tab 921 has an assembled device tab length 971 that can be range from 0.25-6 inches and the tab width 965 that can be a range from 0.25-2 inches. The assembled device tab 921 is located a suitable assembled device tab distance 970 from the open end of tube 930 and the distance can range from 0.25-4 inches.

The cladding section 910 can have a cladding section width of 960 that can range from 0.75-9 inches and a cladding section length 950 that can range from 0.5-9 inches. The cladding section 910 has a cladding section transition 931 that is designed such that it suitably provides for a connection of stretch section 1110 and cladding section 910.

FIG. 10 shows the absorbent core 510 of the alternative design of FIG. 9. FIG. 10 shows the plan view and side view of absorbent core 510. Absorbent core 510 has an absorbent core width 560 that can range from 0.5-9 inches, a length 561 that can range from 0.75-15 inches and a thickness of 1010 that can be range from 0.25-5 inches. The absorbent core 510 comprises of nonabsorbent retainer 530 and absorbent filler 545.

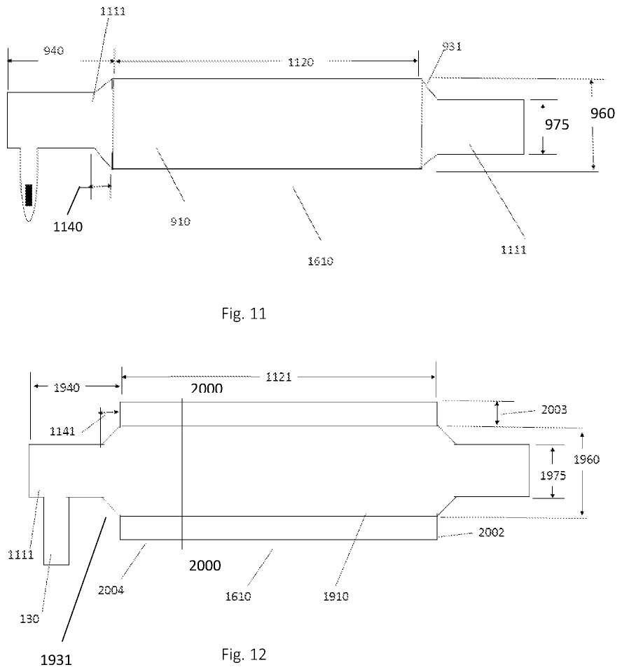

FIG. 11 shows a plan view of the cladding layer 610 that forms the outer layer of cladding section 910 and stretch section 1110. Transition 931 is designed to form a suitable connection to stretch section 1110 and cladding section 910 and is anticipated to have transition length 1140 that can range from 0.1-5 inches. Stretch section 1110 can have a stretch section length 940 and stretch section width 975. The cladding layer 610 has a length 1120 that can range from 1-15 inches and a stretch section width 975 that can range from 0.25-12 inches.

FIG. 12 shows an alternative cladding layer 1610 that is formed from the alternative outer layer of alternative cladding section 1910 and alternative stretch section 1111. The alternative transition 1931 is designed to form a suitable connection to alternative stretch section 1111 and alternative cladding section 1910 and is anticipated to have transition alternative length 1141 that can range from 0.1-5 inches. Alternative stretch section 1111 can have an alternative stretch section length 1940 and alternative stretch section width 1975. The alternative cladding layer 1610 has an alternative cladding layer length 1121 that can range from 1-15 inches and alternative stretch section width 1975 that can range from 0.25-12 inches. The alternative cladding layer 1910 forms a pleat 2001 shown in FIG. 15 that allows the alternative outer layer of cladding section 1910 to open and close. This opening and closing action form a channel that allows urine or discharge from the penis to travel up the alternative cladding layer 1610 and spread to the absorbent core 510. This prevents the urine or discharge from the penis to fill the absorbent core 510 evenly and mitigate the problem of urine or discharge from the penis leaking from the open proximal end 120. Half pleat 2002 has a width of 2003 and edge 2004. The half pleat 2002 forms one half of the full pleat 2001 as shown in FIG. 15.

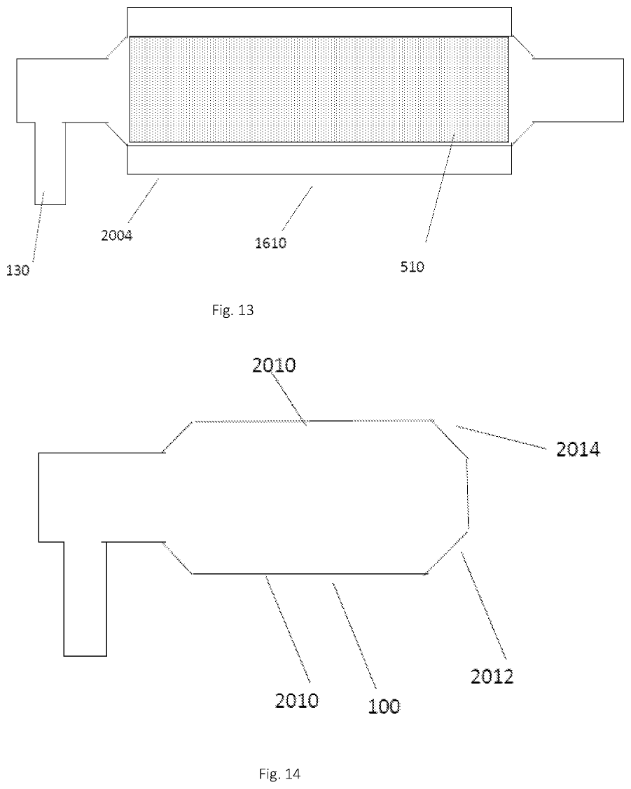

FIG. 13 shows the alternative cladding layer 1610 and the absorbent core 510. The absorbent core 510 is attached to the alternative cladding layer 1610 by using a suitable sealing method such as an adhesive. One can see edge 2004 and at least one retaining device 130.

FIG. 14 shows the alternative incontinence device 100 folded over and seams 2010 sealed. Note the alternative incontinence device 100 is trimmed to form champers 2012 and 2014 on the end and sealed to form a watertight enclosure for the incontinence device 100. The sealing can be achieved by any suitable means such as adhesive, ultrasonic bonding, or heat sealing.

FIG. 15 shows a side view at section 2000-2000 in FIG. 12 showing incontinence device 100 with the pleat 2001 formed by cladding layer 1610 by sealing the edged 2004 shown in FIG. 12 of half pleat 2002 along the edges 2004 using adhesive 2005. The cladding layer 1610 encloses the absorbent core 510. The pleat forms a channel that allows urine or liquid to move either up or down the incontinence device 100 so that the absorbent core 510 can be uniformly filled.

FIG. 16 shows a line drawing perspective view of the incontinence device 100 with closed distal end 110, open proximal end 120 and at least one retaining device 130. Open proximal end 120 has internal diameter 150 and the size of internal diameter 150 depends on the size of the penis to be used with the instant invention and the diameter can be selected from any size including and between 0.5 and 5 inches.

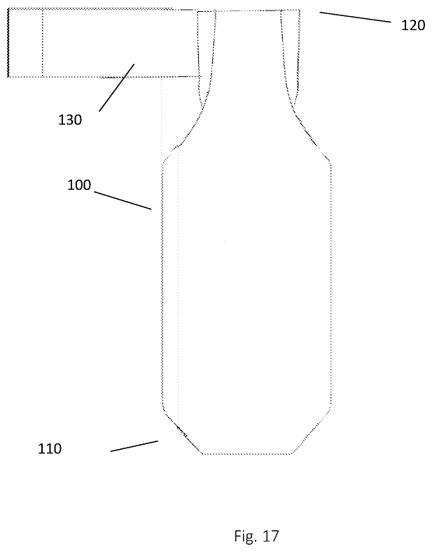

FIG. 17 shows a line drawing plan view of the incontinence device 100 with closed distal end 110, open proximal end 120 and at least one retaining device 130.

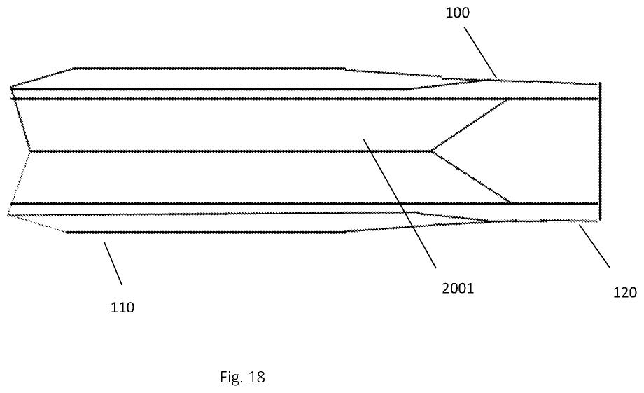

FIG. 18 shows a line drawing side view of the incontinence device 100 with closed distal end 110, open proximal end 120 and pleat 2001.

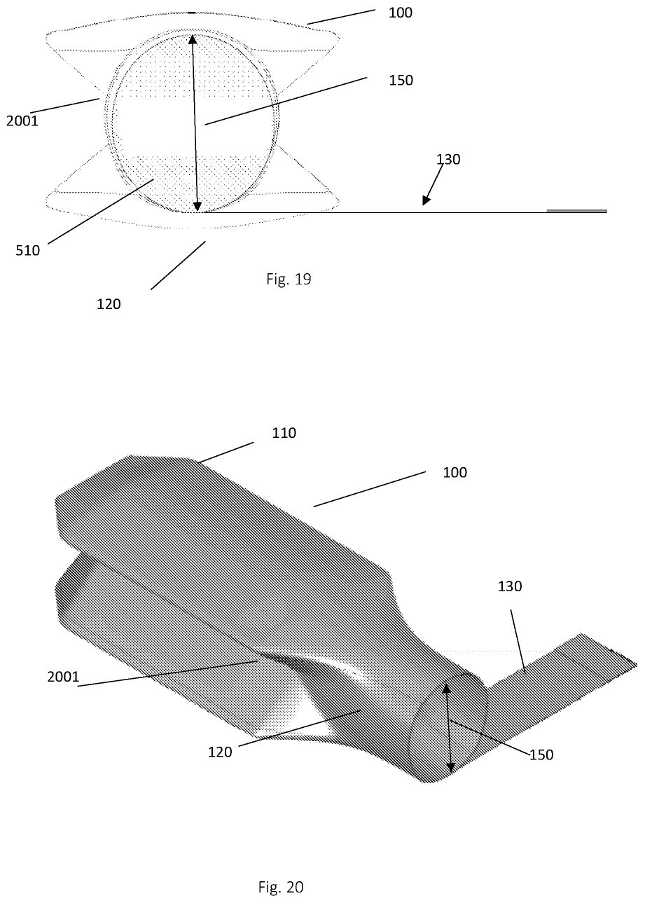

FIG. 19 shows a line drawing end view from open proximal end 120 of the incontinence device 100, showing pleat 2001, diameter 150, at least one retaining device 130 and absorbent core 510.

FIG. 20 shows a rendering of a perspective view of the incontinence device 100 with closed distal end 110, open proximal end 120 and at least one retaining device 130. Open proximal end 120 has internal diameter 150 and the size of internal diameter 150 depends on the size of the penis to be used with the instant invention. The pleat forms a channel that allows urine or liquid to move either up or down the incontinence device 100 so that the absorbent core 510 can be uniformly filled.

Since. modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

I claim:

1. An incontinence device for males, comprising:
a. a tubular body having a closed distal end and an open proximal end for receiving a penis;
b. at least one retaining device for securing the tubular body to the penis; and
c. an absorbent core internal to the tubular body for absorbing urine.

2. The incontinence device of claim 1, wherein the at least one retaining device comprises a fastener selected from the group consisting of adhesive tape, stretch tape, hook, and loop fastener.

3. The incontinence device of claim 1, wherein the absorbent core comprises a nonabsorbent retainer and an absorbent filler.

4. The incontinence device of claim 3, wherein the absorbent filler comprises a mixture of superabsorbent polymer (SAP) and villous pulp.

5. The incontinence device of claim 1, wherein the tubular body is formed from a cladding layer comprising a non-porous material.

6. The incontinence device of claim 1, further comprising a plurality of retaining devices for securing the tubular body to the penis.

7. The incontinence device of claim 1, wherein the tubular body is configured to hold a volume of urine ranging from minor leakage events to overnight leakage events.

8. The incontinence device of claim 1, wherein the tubular body is configured to remain in position during sleep and during moderate to strenuous physical activity.

9. The incontinence device of claim 1, wherein the tubular body is configured to prevent leakage of urine during use.

10. The incontinence device of claim 1, wherein the tubular body is configured to be disposable after use.

11. The incontinence device of claim 1, wherein the tubular body is configured for multiple uses with a washable or replaceable absorbent core.

12. An incontinence device for males, comprising:
a. a tubular body having a closed distal end and an open proximal end for receiving a penis;
b. at least one retaining device for securing the tubular body to the penis;
c. an absorbent core internal to the tubular body for absorbing urine; and
d. said tubular body is configured to be disposable after use.

13. The incontinence device for males of claim 12 wherein the tubular body is selected from the group consisting of latex, silicone, and a flexible plastic material.

14. The incontinence device of claim 12, wherein the at least one retaining device comprises a fastener selected from the group consisting of adhesive tape, stretch tape, hook, loop fastener, straps, belts, buckle, a snap and magnetic closure device.

15. The incontinence device of claim 12, wherein the absorbent core comprises a nonabsorbent retainer and an absorbent filler and said absorbent filler is selected from the group comprising cotton, cellulose, and a superabsorbent polymer.

16. The incontinence device of claim 15, wherein the absorbent filler comprises a mixture of superabsorbent polymer (SAP) and villous pulp, rayon, and bamboo fiber.

17. The incontinence device of claim 12, wherein the tubular body is formed from a cladding layer comprising a non-porous material and said cladding layer is made from a breathable material to promote comfort and reduce skin irritation.

18. The incontinence device of claim 12, further comprising a plurality of retaining devices for securing the tubular body to the penis and said retaining devices are adjustable to accommodate different penis sizes and shapes.

19. The incontinence device of claim 12, wherein the tubular body is configured to hold a volume of urine ranging from minor leakage events to overnight leakage events and said tubular body may include a graduated scale to indicate the volume of urine collected.

20. The incontinence device of claim 12, wherein the tubular body is configured to remain in position during sleep and during moderate to strenuous physical activity.
